(19) United States
(12) Patent Application Publication
(10) Pub. No.: US 2009/0209013 A1
(43) Pub. Date: Aug. 20, 2009

(54) Burkholderia rhizoxina micro-organisms, novel endosymbionts of rhizopus sp. and method for producing rhizoxin and/or rhizoxin-derivates using said micro-organisms

(75) Inventors: Christian Hertweck, Leipzig (DE); Laila P. Partida-Martinez, Jena (DE)

Correspondence Address: JORDAN AND HAMBURG LLP, 122 EAST 42ND STREET, SUITE 4000, NEW YORK, NY, 10168, US

(73) Assignee: Leibniz-Institut fuer Naturstoff-Forschung und Infektionsbiologie e.V. Hans-Knoell-Institu-(HKI), Jena (DE)

(21) Appl. No.: 11/921,698

(22) PCT Filed: Jun. 1, 2006
(86) PCT No.: PCT/DE2006/000962

§ 371 Date: Dec. 5, 2007

(30) Foreign Application Priority Data
Jun. 6, 2005 (DE) DE 102005026417.4

(51) Int. Cl. C12P 17/18; C12N 1/20

(52) U.S. Cl. 435/119; 435/252.1

(57) ABSTRACT

The invention relates to a method for producing rhizoxin and derivates of rhizoxin.

The aim of the invention is to produce novel novel micro-organisms that synthesise rhizoxin or derivates of rhizoxin, and to provide a method for producing rhizoxin oder derivates thereof, without the disadvantages of prior art and in a simpler manner with a higher yield. To this end, bacterial symbionts of Rhizopus sp., in the form of Burkholderia Rhizoxina DSM 17360 or other Burkholderia Rhizoxina strains, are isolated, cultivated and used for the fermentive production of rhizoxin and/or derivates of rhizoxin in a submerged culture, and the rhizoxin and/or the derivates thereof are subsequently isolated from the culture supernatant.

### BACKGROUND OF THE INVENTION

The invention relates to burkholderia rhizoxina micro-organisms, novel endosymbionts of rhizopus sp. and to the method for producing rhizoxin and/or derivates of rhizoxin using said micro-organisms.

Rhizoxin [(1) in Table 1] is a polyketide metabolite with an antifungal and cytotoxic effect that has been isolated from different fungi of the rhizopus genus (e.g. rhizopus microsporus) (J. Jennessen et al. 2005, J. Agric. Food Chem. 53: 1833-18409).

The cytoxic power of rhizoxin is caused by a very effective bond to β-tubulin that leads to the inhibition of mitosis (Y. Koga-Ban et al. 1995, DNA Res. 2: 21-26; M. Takahashi et al. 1987, Biochim. Biophys. Acta 926: 215-223).

Due to this anti-miotic activity that has been observed in most of the eucaryotic cells, particularly in different human tumor cell lines, attention has been focused on rhizoxin as a potential active anti-tumor substance (T. Tsuruo et al. 1986, Cancer Res. 46: 381-385; A. Jordan et al. 1998, Med. Res. Rev. 18: 259-296).

Similar strong anti-fungal and anti-tumor effects have also been observed for the derivates of rhizoxin [(29 bis (10) in Table 1] (S. Kiyoto et al. 1986, J. Antibiot. 39: 762-772; S. Iwasaki et al. 1986, J. Antibiot. 39: 424-429; S. Iwasaki et al., 1986, Chem. Pharm. Bull. 34: 1387-1390).

However, the production of rhizoxin and the derivates thereof by fermentation of rhizopus strains and the purification of the cytotoxic bonds are difficult and complex and the yield of rhizoxin or rhizoxin derivates is low.

### SUMMARY OF THE INVENTION

The aim of this invention is to produce novel micro-organisms that synthesise rhizoxin or derivates of rhizoxin and to provide a method for producing rhizoxin oder derivates thereof, without the disadvantages of prior art and in a simpler manner with a higher yield.

In this invention, the term “derivate” describes variants that have the same functional properties like rhizoxin. The derivates can also have limited functional properties.

Surprisingly it has been found that not the rhizopus sp. strains are the producers of rhizoxin but bacterial endosymbionts burkholderia rhizoxina, which are closely related with species of the burkholderia genus, produce these natural substances and that said bacterial endosymbionts of rhizopus sp. are useful to simplify the production of rhizoxin and/or the derivates thereof with a higher yield in a liquid culture.

### DESCRIPTION OF THE INVENTION

According to this invention, the bacterial endosymbionts are isolated from rhizoxin producers, such as rhizopus microsporus van Tieghem var. chinensis (ATCC 62417), rhizopus sp. F-1360 (ATCC 20577), rhizopus microsporus var. microsporus (CBS 699.68 and CBS 308.87 strains), by cultivating the rhizopus strains in a liquid medium (1% cornstarch, 0.5% glycerol, 1% flour of gluten, 1% yeast, 1% cornsteep, and 1% CaCO₃-pH 6.5) or in malt extract-pepton medium at 30° C. under stirring with 120-200 rpm, by transfering the mycelium of a growing rhizopus culture (e.g. 20 ml) in a vessel (e.g. a 50 ml vessel) afterwards and by splitting it mechanically in a pipetting procedure and by digesting it mechanically.

The digested cells have been subject to a gradient centrifugal process (e.g. 500 rpm during 5 min, 4000 rpm during 20 min).

After each centrifuging step, aliquots of the supernatant are spread on agar plates (5.0 g pepton, 3.0 g meat extract, 15.0 g agar, 1000 ml water) and incubated at 30° C. The separated colonies of the endosymbiont can be isolated from the plates as pure cultures after the cultivation.

For example, the endosymbiont strain DSM 17360 (Burkholderia rhizoxina) can be isolated.

The burkholderia rhizoxina strain (DSM 17360) was deposited as this designation in the “Deutschen Sammlung für Mikroorganismen und Zellkulturen GmbH (DSMZ)” (German Collection for Microorganisms and Cell Cultures) in Braunschweig, Mascheroder Wege 1.

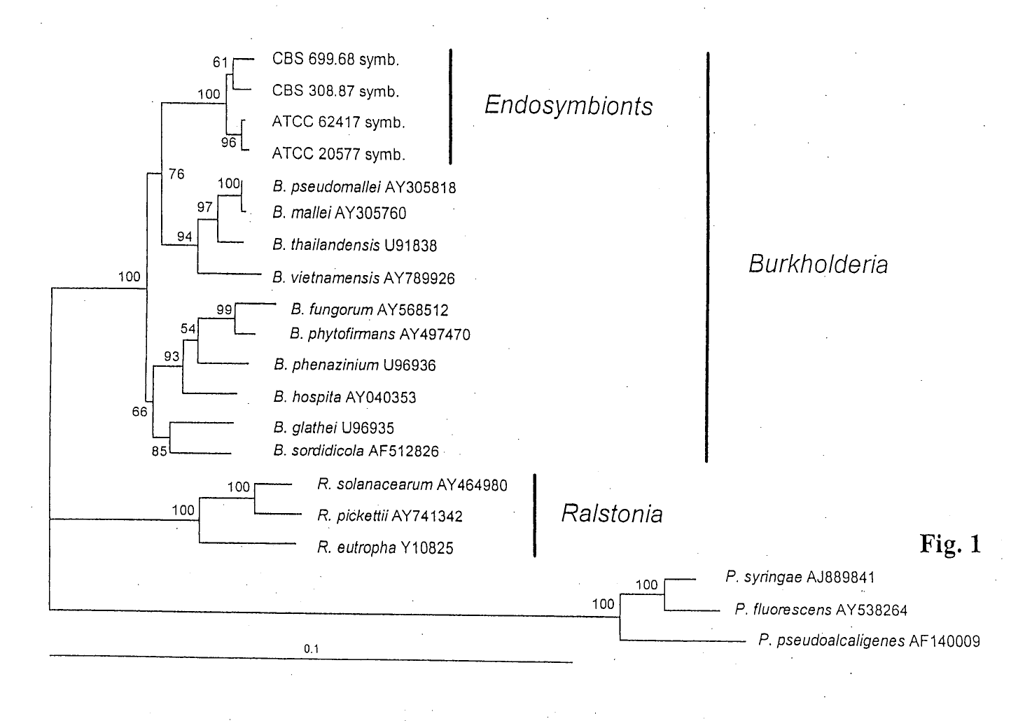

The DSM 17360 endosymbiont strain differs from the strains known so far in its 16S r-RNA sequence as shown in the phylogenetic illustration in FIG. 1.

According to this invention, the bacterial colonies obtained in this manner (burkholderia rhizoxina strains) can be used for the production of rhizoxin and of the derivates of rhizoxin in the following way:

The bacteria strains are fermented as a submerged culture in a liquid medium (1% cornstarch, 0.5% glycerol, 1% flour of gluten, 1% yeast, 1% cornsteep and 1% CaCO₃-pH 6.5) at 30° C. or 5.0 g pepton and 3.0 g meat extract in 1000 ml water at 30° C.

The rhizoxin or the derivates thereof can be gained from the culture supernatant by using organic solvents for extraction, such as ethyl acetate. Alternatively, known methods can be applied for the solid phase extraction.

Here, the yield of rhizoxin or the derivates of rhizoxin ranges between 10 mg/l and more than 200 mg/l culture supernatant.

1. A microorganism in the form of Burkholderia rhizoxina, deposit DSM 17360.

2. Use of the burkholderia rhizoxina micro-organism of claim 1 for producing rhizoxin or a rhizoxin derivative selected from the following structures:

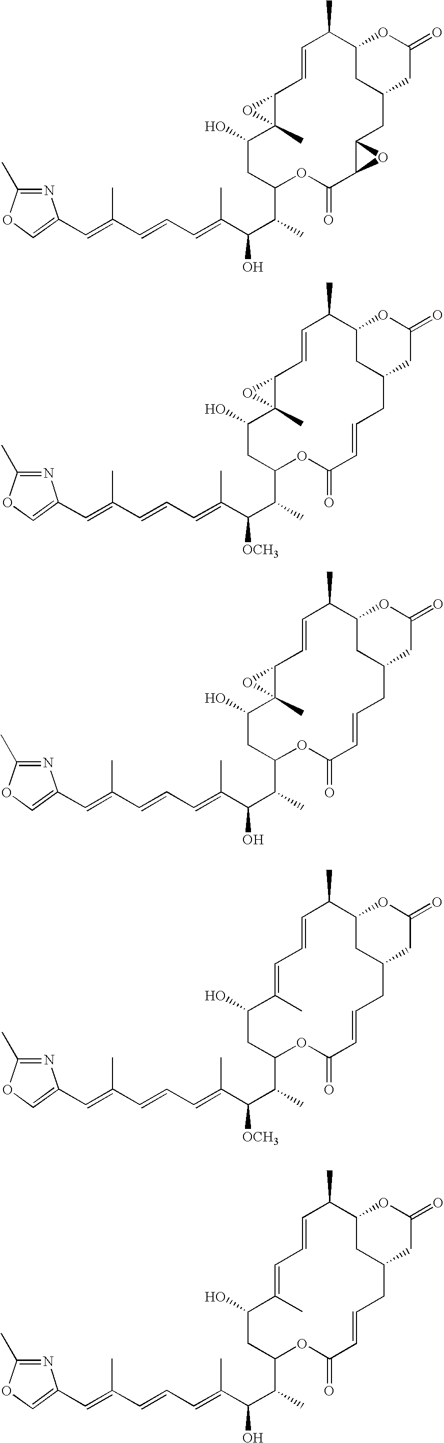

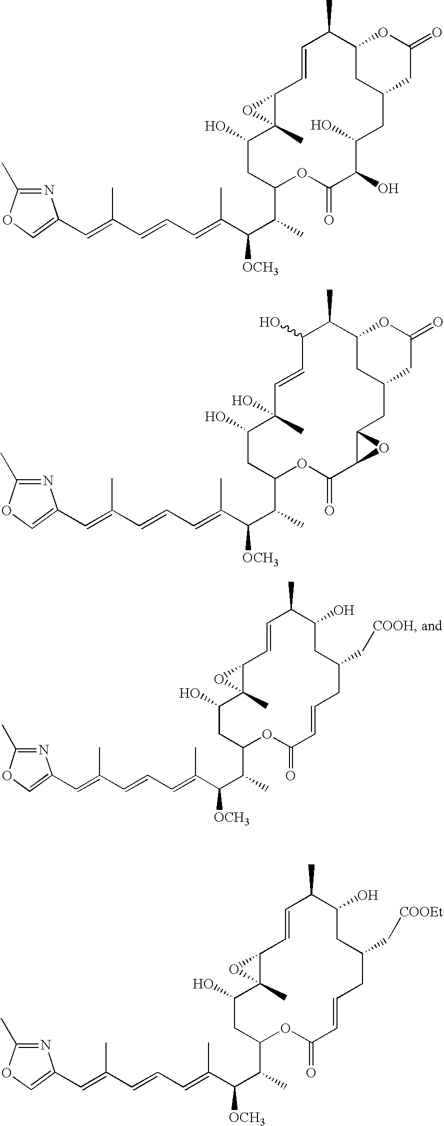

3. A method for producing rhizoxin or a derivative of rhizoxin selected from the following structures: 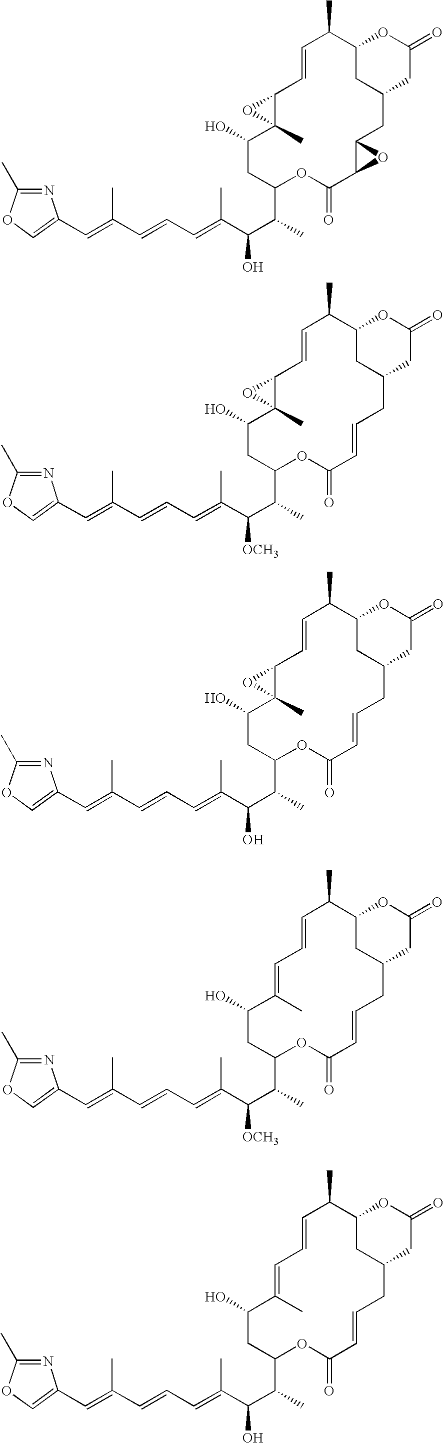 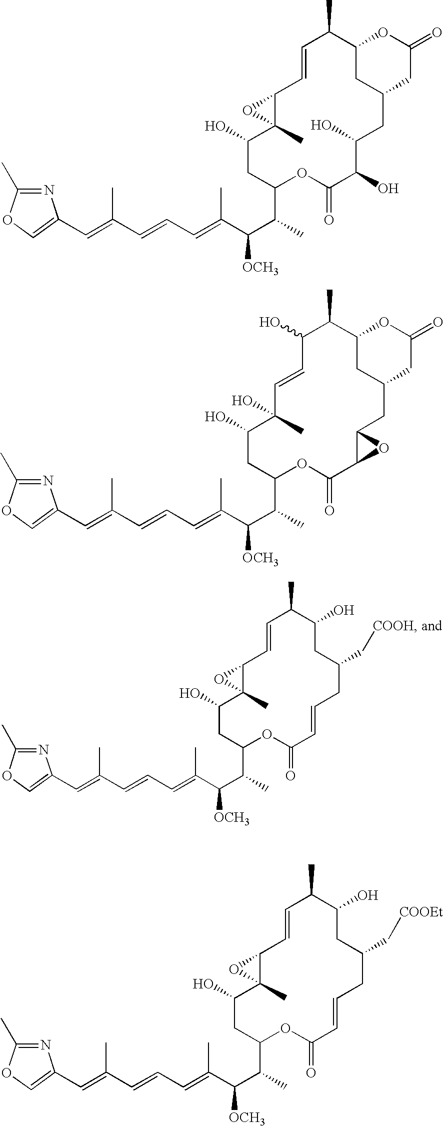
the method comprising the steps of cultivating, in a culture medium, a bacterial symbiont from rhizopus sp. in form of a burkholderia rhizoxina strain in submerged culture until rhizoxin or the derivative of rhizoxin accumulate in the culture supernatant and are isolated from the culture supernatant.

4. The method of claim 3, wherein the burkholderia rhizoxina strain is DSM 17360.

5. The method of claim 3, wherein the culture medium contains 1% cornstarch, 0.5% glycerol, 1% flour of gluten, 1% yeast, 1% cornsteep and 1% CaCO₃.

6. The method of claim 3, wherein the culture medium is a malt extract peptone medium.

7. The method of claim 5, wherein the pH-value of the culture medium is 6.5.

8. The method of claim 5, wherein the fermentation temperature is 30° C.

9. The method of claim 5, wherein, during cultivation, the culture medium is agitated at 120-200 rpm.

10. The method of claim 4, wherein the rhizoxin or the rhizoxin derivative is isolated from the culture supernatant by means of extraction using organic solvents or adsorption resins.
